(19) United States
(12) Patent Application Publication
(10) Pub. No.: US 2003/0044474 A1
(43) Pub. Date: Mar. 6, 2003

(54) High molecular weight, lipophilic, orally ingestible bioactive agents in formulations having improved bioavailability

(75) Inventors: Kar Wai C. Tao, Redwood Shores, CA (US); Ping Yu, Fremont, CA (US); Richard L. Roberts, Germantown, TN (US)

Correspondence Address: KLARQUIST SPARKMAN, LLP, 121 SW SALMON STREET, SUITE 1600, PORTLAND, OR, 97204, US

(73) Assignee: Shaklee Corporation

(21) Appl. No.: 10/080,975

(22) Filed: Feb. 21, 2002

Related U.S. Application Data
(60) Provisional application No. 60/310,151, filed on Aug. 3, 2001.

(51) Int. Cl. A61K 35/78

(52) U.S. Cl. 424/729; 424/757; 424/725; 424/766; 424/732

(57) ABSTRACT

Orally ingestible bioactive agents are disclosed which contain a triglyceride matrix and one or more polyphenols that improve the bioavailability of the bioactive agent. In particular non-limiting examples, the bioactive agent is a ubiquinone (such as Coenzyme Q), the triglyceride matrix is a soybean oil matrix, and the composition further includes additional anti-oxidants.

The present application claims priority from U.S. provisional application Serial No. 60/310,151, filed on Aug. 3, 2001.

### FIELD OF INVENTION

The present technology relates to improved compositions and methods for achieving bioavailability and/or stability of large, high molecular weight, lipophilic, bioactive agents in orally ingested compositions, and to methods for the preparation of such compositions.

### BACKGROUND OF THE INVENTION

The ability to orally deliver adequate quantities of large, high molecular weight, lipophilic, bioactive (for example therapeutic) agents, such as dietary and pharmaceutical ingredients, has presented problems for scientists involved in the formulation of such products. Because of their size, molecular weight, and lipophilic (hydrophobic) nature, these agents are not soluble in aqueous media. Additionally, their solubility is not significant in either gastric fluids or even in bile fluids. Therefore, because of their inherent lack of solubility in aqueous media, these important agents are not readily absorbed in the digestive tract of the human body. Although these agents are soluble in lipids, they show poor bioavailability when administered in the form of an oil solution or in any form of water and oil suspension or emulsion. Therefore a low concentration or a prolonged build-up in the systemic circulation is often required.

Traditional methods of ensuring adequate delivery of such lipophilic bioactive agents to the human body have involved two primary paths. First, these agents have been formulated into products in such an amount that there is a significant excess of the bioactive agent compared to the amount required for the desired biological activity in order to achieve the desired blood levels. Secondly, such bioactive agents have been administered in multiple doses to be taken throughout the day so that a smaller excess of the agent is required in each dose compared to a single dose. Even in the latter case, a significant excess of the bioactive agent is required to achieve the desired biological activity. Perhaps the most important aspect of either of these methods is that the excess bioactive agent can cause gastrointestinal distress. For some bioactive agents, this excess dosage can potentially be toxic. Additionally, since these bioactive agents are often expensive, the required excess of the agent can increase the cost per dose when compared to the amount of the bioactive agent needed to achieve the desired effectiveness of the product.

A large, high molecular weight bioactive agent is an agent having a biological activity, and which has a molecular weight of at least 200, for example at least 300 or 400. Examples of the class of large, high molecular weight, lipophilic nutritional and pharmaceutical bioactive agents include therapeutic agents that are designed to achieve a therapeutic (including a nutritional) result, such as steroids (for example estrogens such as 17-beta-estradiol, or androgens such as testosterone, or their biological precursors), steroid antagonists, non-steroidal anti-inflammatory agents (NSAIDS such as ibuprofen), antihypertensive agents (such as methyldlothiazide), antioxidants (such as Vitamin A or Vitamin C), anti-seizure agents (such as lorazepam or primidone), antibiotics (such as amphotericin B, clarithromycin, erythromycin, nystatin, or clotrimazol), antiviral agents, anticancer agents (such as docetaxel, etoposide, lomustine, paclitaxel, or teniposide), neuroprotective agents (dexanabinol), antidepressive agents, enzymes, coenzymes, proteins, globulins, vitamins, retinoids, immunologicals, nucleotides, lectins, growth factors, etc. Although any of the compounds represented by these classes could have been chosen as a example of the technology to be described herein, Coenzyme Q10 (Ibiquinone or CoQ10) was chosen because the state of the formulation art described in the numerous available published papers and patents demonstrates the utility of the invented technology.

Coenzyme Q10 (CoQ10) is an important biological molecule which has the chemical name 2,3-dimethoxy-5-methyl-6-decaprenyl-1,4-benzoquinone, and is a member of the class of ubiquinones (which are a group of lipid soluble benzoquinones involved in electron transport in the mitochondria). The total quantity of CoQ10 in the human body is estimated to be 1.4 to 1.8 grams, depending upon the age and physical condition of the individual. Because it is found in the mitochondria and some other cellular organelles of every living cell, it is most abundant in cells that actively consume energy, such as the cells of the skeletal muscles and the heart. Blood acts as a reservoir and transport medium for CoQ10 after the CoQ10 is endogenously synthesized in the liver or is exogenously acquired through intestinal absorption from digested food substances. It is estimated that endogenous synthesis of CoQ10 accounts for approximately 55% of the human biological requirement. Therefore, the remaining 45% must be obtained either from consumed food or through nutritional supplementation. As humans age, the endogenous synthesis of CoQ10 drops dramatically. Therefore, there is an even greater need for supplementation in the elderly, especially in those who have certain diseases such as mitochondrial myopathy, and those taking drugs such as those to lower cholesterol (the so called “statin” drugs) that deplete the endogenous CoQ10 levels in the body.

CoQ10 has a molecular weight of 864. Because of its size and structure, it is very lipophilic, practically insoluble in water, and soluble in a limited number of oils. Additionally, it is readily recognized that CoQ10 is very insoluble in normal human/animal digestive fluids, thereby resulting in its poor bioavailability from oral dosage forms. Because of its high molecular weight and lipophilic nature, this molecule is only slowly absorbed into the intestinal tract. Furthermore, since it is absorbed through the microvilla lacteal, its appearance in the blood stream is significantly delayed compared to smaller water soluble molecules which are readily absorbed into the vascular system. Furthermore, since CoQ10 melts at a temperature that is 10° C. above normal body temperature, and digestive fluids cannot readily dissolve the dry powder form of this nutrient, the dry powder is virtually not absorbed by the microvilla lacteal. Therefore, any technology that markedly enhances uptake of CoQ10 represents a significant advance in the delivery of this molecule to the human body. Since CoQ10 is a good general representation of the class of large, high molecular weight, lipophilic bioactive agents, any technology that results in its enhanced bioavailability has application to other bioactive agents in this class.

A variety of methods have been investigated to reduce the dosage quantities and/or the dosage frequency of CoQ10. Perhaps the oldest methods involve the administration of such therapeutic agents in oily preparations, for example dissolving the therapeutic agent in neutral oils, such as castor oil, or as mixtures of such oils with high molecular weight polyols such as polyglycerol. A preparation of this type is described in U.S. Pat. No. 4,156,718, but such preparations are unpleasant to administer because of their odor and taste, as well as the fact that many lipophilic bioactive agents have an undesirable and/or bitter taste themselves. Additionally, such oily preparations have a tendency to coat the mouth and thereby further reduce patient compliance and inhibit consumption of such preparations. Furthermore, because such formulations are not readily broken down by the digestive system, the CoQ10 dissolved in these formulations tends to pass through the digestive system without being released from the oleaginous matrix in which it is ingested. Therefore the bioavailability of the agent is not significantly improved by its incorporation into such a matrix.

The administration of CoQ10 in soybean oil via oral administration was disclosed by K. Folkers and K. Muratsu (Biomedical and Clinical Aspects of Coenzyme Q, Volume 3, K. Folkers and Y. Yamamura eds., Elsevier/North-Holland Biomedical Press, Amsterdam, 31-42, 1981). That publication described a soft gel capsule containing 33.3 mg of CoQ10 in about 400 mg of soybean oil. This method represented some improvement in the oral delivery of CoQ10, but it suffered from problems with long-term shelf life because the CoQ10 would crystallize out of the soybean oil, thereby limiting the bioavailability of this bioactive agent.

An early use of a neutral oil to dissolve the CoQ10 is found in U.S. Pat. No. 4,824,669, which describes the formation of a stable emulsion capable of delivering CoQ10 to the human body by intravenous administration. The vehicles for intravenous administration were soybean, corn, peanut, safflower, or olive oil emulsions into which the CoQ10 was dissolved. This method improves delivery of CoQ10 to the body, but it is confined to the intravenous administration of this large, high molecular weight, lipophilic, bioactive agent.

In addition to solutions of CoQ10 in oils and high molecular weight glycerols, clear micellized solutions have been employed to deliver CoQ10. U.S. Pat. No. 4,572,915 describes a method for producing such clear, micellar solutions of fat soluble vitamins and essential nutrients that permit enhanced absorption of those vitamins and nutrients. Specifically, this patent describes a method for delivering vitamins such as fat soluble vitamins (such as Vitamins A, E, D, and/or derivatives), essential nutrients, non-water soluble drugs, medicinal and pharmaceutical agents, in a mixture of polyethoxylated castor oil (such as the 30 and 40 mole ethoxylated castor oils) and a pharmaceutically acceptable polyol (such as glycerol or diethylene glycol) which when heated above 55° C. in either the presence of (or absence) of water forms a uniform homogeneous mixture that can be diluted with water.

A more recent formulation technology involves the mixture of bioactive agents into solid lipophilic oral dosage forms. This method, as described in U.S. Pat. No. 5,989,583, involves mixing at least one solid fat and a phospholipid with the bioactive agent. The mixture is then delivered to the organism in an appropriate dosage form such as a gelatin capsule, a tablet, or even a beverage. Specifically, the fat described in this patent is either a triglyceride or mixture of triglycerides, and the phospbolipid is lecithin. The bioactive agent, triglyceride, phospholipid and an antioxidant are dissolved in a solvent such as dichloromethane. The solvent is evaporated to complete dryness and the lipid mixture is then hydrated with water by mechanical shaking. The resultant lipid dispersion is then homogenized with a high-pressure homogenizer to reduce the particle size to the submicron range. This bioactive-lipid preparation is then mixed with a cryoprotectant such as sucrose and a flow-imparting agent, freeze-dried, and placed in capsules. This type of formulation, which involves multiple steps and solvents and must be handled carefully because of environmental concerns, is no longer economically feasible. Additionally, the enhanced bioavailability achieved is only moderate, especially in view of the expense involved and the complexity of the formulation.

An alternative method involves a formulation containing the bioactive therapeutic agent in a matrix containing a solubilizing agent and an edible polyhydric alcohol to create a liquid formulation that is encapsulated in a gelatin capsule as set out in U.S. Pat. No. 6,056,971. The bioavailability of the CoQ10 from this formulation was said to be greater than a formulation of the CoQ10 dissolved in a standard vegetable oil vehicle (the “reference” CoQ10 capsules). The difficulty with this type of formulation is that it is composed of almost 90% solubilizing agent that is selected from a group of non-ionic surface-active agents. As long as food grade materials are used in the formulation, these materials are not generally considered to be harmful when ingested. However, the ingestion of the amount of surface-active agents needed to achieve enhanced CoQ10 bioavailability can soften stools or even cause diarrhea. Additionally, for the reasons described above, it is not difficult to demonstrate enhanced bioavailability of a formulation compared to the bioavailability of the same large, high molecular weight, lipophilic bioactive agent dissolved in a standard vegetable oil since the delivery of such agents from the latter matrix is extremely poor.

An alternative method as described in U.S. Pat. No. 6,191,172 involves a formulation containing a bioactive agent and a solubilizing agent created by chemically combining a tocopherol or sterol derivative (such as a sebecate) with high molecular weight polyethylene glycol or methoxypolyethylene glycol. Although no data is presented to demonstrate the enhanced bioavailability of CoQ10 from this formulation, the bioavailability of the patented technology was compared to that of CoQ10 in an oil formulation. As discussed above, it is not difficult to demonstrate enhanced bioavailability of a formulation compared to the bioavailability of the same large, high molecular weight, lipophilic bioactive agent dissolved in a standard vegetable oil because the delivery of bioactive agents from the latter matrix is poor. Additionally, the patent describes toxicity issues with one of the chemically combined tocopherol-polyethylene glycol-sebecate solubilizing compounds. Since this derivative is the commercially available molecule, there is an indication that this technology needs significantly more research effort before it can be considered to be a commercially viable method for enhancing the bioavailability of large, high molecular weight, lipophilic bioactive agents.

U.S. Pat. No. 6,184,255 describes a novel way of improving the bioavailability of CoQ10 by administering a combination of the oxidized and reduced forms of this bioactive agent. This patent teaches that the bioavailability of the agent is less dependent upon the medium in which the agent is delivered, but more importantly, is dependent upon the oxidation state of the agent. Although this may be true, the ability to obtain and stabilize a mixture of the oxidized (Ubiquinone) and reduced (Ubiquionol) forms of CoQ10 is significantly more difficult than is apparent. The reduced form of CoQ10 is obtained by reacting the oxidized form with electron donating compounds such as sodium borohydride or sodium dithionite (sodium hydrosulfite). Since oxygen in air has the potential to react with the reduced form, it can readily be reconverted to the oxidized form upon standing. Therefore, this technology requires the presence of significant amounts of antioxidants to stabilize the amount of the reduced form of CoQ10 present in the mixture throughout the manufacturing process as well as during the storage of the oral dosage form. Therefore, although theoretically feasible, this method of enhancing bioavailability is of limited commercial value.

Polyphenolic compounds are readily found in many foods and herbs, and they are commonly found in nature. Tea, particularly green tea, is rich in polyphenolic compounds. Similarly, grapes (particularly purple grapes) and beverages such as wine made from grapes (particularly red wines) contain a significant number of polyphenolic compounds. These materials have been previously used as antioxidants for a variety of purposes. Patents citing the use of polyphenolic compounds for their antioxidant activity include U.S. Pat. Nos. 5,648,377; 5,985,300; 6,013,32; 6,046,181; 6,107,281; and 6,162, 419.

Compositions containing polyphenolic compounds have been the subject of other patents, such as U.S. Pat. Nos. 6,086,910 and 6,099,854, that describe the use of these materials for use in food supplements for the improvement of blood lipid profiles, especially the reduction of low-density lipoproteins (LDL). The '854 patent mentions including CoQ10 in the composition as an antioxidant.

U.S. Pat. No. 5,827,886 describes the use of a composition for the relief of arthritis-induced symptoms from the topical application of a composition that could contain polyphenolics as antioxidants.

Finally, U.S. Pat. No. 6,063,820 describes a medicinal food for diabetics that could contain CoQ10 and a polyphenolic compound (specifically resveratrol is mentioned) as antioxidants in the preparation.

### SUMMARY OF THE DISCLOSURE

It has been discovered that polyphenolic compounds can increase the absorption of a large, high molecular weight, orally ingested, lipophilic bioactive agent or combination of bioactive agents when these materials are simultaneously administered from a triglyceride matrix. This combination of the polyphenolic compound with the bioactive agent in an oil matrix also increases the shelf life of the preparations of the present invention because it prevents the crystallization of the bioactive therapeutic agent from the triglyceride matrix.

### BRIEF DESCRIPTION OF THE FIGURES

FIG. 1 is a structural core formula of a flavone.

FIG. 2 is a structural core formula of a flavonol.

FIG. 3 is a structural core formula of an anthrocyanin.

FIG. 4 is a structural formula of resveratrol.

### DETAILED DESCRIPTION OF THE INVENTION

Explanation of Terms:

Antioxidant: An agent that inhibits oxidation. Examples are Vitamin A and Vitamin C.

Bioactive agent: An agent (such as an oral dosage preparation) having a biological activity. An example of a bioactive agent is a therapeutic agent, which is administered to maintain health, inhibit its deterioration, or treat or inhibit a pathological condition. Some bioactive agents may be other than therapeutic agents, for example diagnostic agents. Nutritional supplements are examples of bioactive agents.

High Molecular Weight: Having a molecular weight of at least 200. In specific examples, high molecular weight agents will have a molecular weight of at least 300, 400, 500, 800, 1000 or more.

Lipophilic: Tending to dissolve in lipid and non-polar solvents, but is sparingly soluble to insoluble in water. Lipophilicity can be measured by a tendency to segregate with lipids in a water/oil mixture. Highly lipophilic substances have an octanol/water partitioning coefficient of 4 or more. This partitioning coefficient demonstrates the greater organic solvent solubility, because octanol is an organic solvent with low polarity. In some particular examples, the partitioning coefficient is 5 or more, or 6 or more.

Liquid: A flowable substance, including liquid gels or oils. The term “liquid” does not include a dry powder, but it does include substances that are administered as liquids (for example a gel oil), or become liquids at human body temperature.

Low polarity molecule: A molecule that does not possess sufficient ionizable groups in its structure so as to make it significantly soluble in water. This includes, for example, a molecule that does not include any ionizable groups (for example a hydrocarbon).

Pharmaceutically acceptable salts and esters: Salts (such as sodium or potassium salts or esters (such as acetates and carbonates), which are biologically compatible. Bioactive agents described herein include salts, esters, and other biologically compatible derivatives, unless indicated otherwise.

Soybean lipid: A lipid (or mixture of lipids) obtained from soybean.

Ubiquinone: A biologically ubiquitous lipid soluble benzoquinone involved in electron transport in mitochondria. Ubiquinone structures are based on the 2,3-dimethoxy-5-methyl-benzoquinone nucleus with a variable terpenoid side chain containing one to twelve mono-unsaturated trans-isoprenoid units. The nomenclature of this class of compounds is Qx, wherein x is the total number of isoprenoid (—CH₂—CH═C(CH₃)—CH₂—) units in the side chain. Naturally occurring examples are Coenzymes Q6-10, although an entire series (including Q50) has been synthesized.

As used in this specification, the singular includes the plural, unless the context clearly indicates otherwise. Hence “a,” “an” or “the” can include both the singular and the plural. For example, reference to “a” polyphenol includes either a single polyphenol or a mixture of polyphenols.

Examples are provided for purposes of illustration and not limitation.

The bioavailability of orally administered, large, high molecular weight, lipophilic bioactive agents are difficult to solubilize in any aqueous-based material. Given this lack of solubility in aqueous systems, it is not surprising that digestive fluids do not solubilize these bioactive agents. Therefore, scientists have spent a significant amount of research effort investigating methods for improving delivery systems for these bioactive agents.

These orally administered, large, high molecular weight compounds include steroids, steroid antagonists, non-steroidal anti-inflammatory agents (NSAIDS), antihypertensive agents, antioxidants, anti-epileptic agents, antibiotics, antiviral agents, anticancer agents, antidepressive agents, enzymes, coenzymes, proteins, globins, vitamins, retinoids, immunologicals, nucleotides, lectins, growth factors, etc. Examples of high molecular weight, lipid soluble bioactive agents that are not highly soluble in water include those in Table 1.

Although any of the compounds represented by these classes could have been chosen as a example of the technology to be described herein, the ubiquinones, and particularly Coenzyme Q10 (Ubiquinone or CoQ10), was chosen because the state of the formulation art described in the numerous available published papers and patents particularly demonstrates the utility of this application.

Ubiquinone 10 (Coenzyme Q-10 or CoQ10) and its oxidized counterpart ubiquinol are classical examples of a large, high molecular weight, lipophilic bioactive agent. These molecules are important nutritional and therapeutic agents for humans as well as animals. However, because of its structure (particularly its decaprenyl side chain), CoQ 10 as a representative of the broad class of large, high molecular weight, lipophilic, bioactive agents, is difficult to make available to the body from conventional oral dosage forms. Therefore, this class of agents is generally given orally in significantly higher doses than that needed for the desired therapeutic activity in order to ensure the maximum possible delivery to the body. Due to the expense of providing such dosage forms, and poor bioavailability of these agents, a significant research effort has been made to identify ways to improve their bioavailability.

It has been discovered that by combining such large, high molecular weight, lipophilic, bioactive agents with a polyphenolic compound (including combinations of polyphenolic compounds), the bioavailability of the bioactive agent is significantly improved. As a class, polyphenolic compounds include any ingredient containing two or more hydroxyl groups on a phenyl ring, especially the dihydroxy or trihydroxyphenyl groups. Polyphenols therefore include compounds that have at least one phenyl ring that has at least 2 or 3 hydroxyl groups on it, and compounds having multiple rings with multiple hydroxyls on each ring. Examples of the class of polyphenols include, but are not limited to, resveratrol, Polygonum cuspidatum extract, green tea extract, grape or grape seed extract, blackberry extract, blueberry extract, cranberry extract, elderberry extract, black current extract. Additionally, the class of polyphenol compounds includes catechins, catechin derivatives (such as epicatechin, epicatechin gallate, etc.), flavanols (such as quercetin, kampferol, and myricetin), flavondiols, theaflavins, thearubigens, anthocyanidins, substituted anthocyanidins, rutins, substituted rutins, tannins, genisteins, and substituted genisteins. This class of compounds includes any polyphenolic compound regardless of whether it is naturally and/or synthetically produced. Polyphenols that are particularly useful for oral ingestion are those that are non-toxic, that is they are suitable for human ingestion without representing a substantial health hazard. The foregoing examples fall into this category of non-toxic agents, that are not recognized health hazards.

It has also been discovered that the presence of the polyphenolic compound (including a combination of such compounds) improves the stability of the bioactive therapeutic compound by preventing crystallization of the bioactive therapeutic compound from the orally ingested dosage form. This prevention of crystallization makes the dosage form stable for longer periods of time.

Although the mechanism of the activity of the polyphenol in combination with the large, high molecular weight, lipophilic, bioactive agents has not been determined, several theories are proposed. Without being limited by those theories, the polyphenolic compounds could be exerting one or more of three primary effects upon the ubiquinone. First, because polyphenolic compounds are known antioxidants, the enhanced bioavailability of CoQ10 could be a result of a reduced oxidation of the CoQ10 in the oil matrix of the soft gelatin capsule. Although this is a possible source of the enhanced bioavailability, it is not likely to be the major explanation for the results found since the inclusion of comparable quantities of tocopherol (Vitamin E), which should also prevent oxidation, does not result in enhanced bioavailability. The second possible source of the enhanced bioavailability is from an interaction between the high molecular weight, orally ingested, lipid soluble bioactive agent(s) and the polyphenolic compound, perhaps via a co-solubilizing effect or by some other form of interaction so that the microvilla lacteal of the small intestine of the digestive system absorbs more of the therapeutic agent from a single dosage. A third possible mechanism for the enhanced absorption is that the polyphenolic compounds are somehow occupying a space in the solution that keeps the agent from forming crystals. This could result from a molecular interaction between the polyphenolic compound and the bioactive agent.

Of the possible mechanisms, the co-solubility or molecular interaction mechanisms are believed to be the primary basis for the enhanced bioavailability of CoQ10, because the data in this specification shows that the CoQ10 is more readily soluble in the oil matrix in the presence of the polyphenolic compounds than in its absence. Furthermore, upon standing for extended periods of time as well as upon exposure to accelerated storage conditions, the CoQ10 remains in solution more readily as witnessed by a significantly reduced growth of crystalline CoQ10. Regardless of the nature of the interaction or the way in which it is achieved, the amount of the bioactive agent absorbed by the digestive system is enhanced by the presence of the polyphenolic compound resulting in higher blood levels of the high molecular weight, lipophilic agent. The data supporting this phenomenon are described below.

In the past 20 years, there have been numerous scientific studies reporting data on the absorption of CoQ10. Some of these studies were conducted on formulations containing a dry powdered form of CoQ10 in conventional two-piece gelatin capsules and others have been on oil based formulations of CoQ10 in single-piece soft gelatin capsules. Table 2 shows the absorption results of a series of studies at a 100 mg dosage of CoQ10 dry powder formulations. It is noteworthy that the peak blood levels for the best of these formulations was found to be 2.44 μg/ml. Table 3 shows a similar set of results for the soybean oil based formulations at a 100 mg dosage of CoQ10. The soybean oil based formulations are superior to the dry powdered formulations because the peak plasma absorption from the soybean oil based formulations is essentially equivalent to or higher then the highest value observed for the dry powder formulation. In fact, the highest value for the oil based soft gel formulation was found to be 2.84 μg/ml, which is significantly higher than those found for the dry powder formulations.

The formulations contain optional anti-oxidants, other than the bioactive agent and/or the polyphenol (both of which are capable themselves of providing anti-oxidant activity). Examples of the additional anti-oxidants include Vitamin A, Vitamin E, Vitamin K, Copper (as cupric oxide), Zinc (as zinc oxide), Iron (as ferrous salt), Selenium (sodium selenate), beta-carotene, catechin, quercetin, eriodictyol, carnosic acid, carnosol, rosmarrinic acid, caffeic acid, coumaric acid, cinnamic acid, Coenzyme Q10, Probucol, astaxanthin, lycopene, alpha-lipoate, and urate, or a pharmaceutically acceptable salt or ester of any such antioxidant

Hence, the present specification discloses an oral dosage composition of a high molecular weight, lipophilic, bioactive agent, in which the composition includes a biologically effective amount of the bioactive agent, a lipid matrix in which the bioactive agent is suspended, and a sufficient amount of a polyphenol to improve gastrointestinal absorption of the bioactive agent when the dosage form is orally administered. In particular examples, the lipid matrix is a triglyceride matrix, such as a soybean lipid matrix, for example a mixture of refined soybean oil, mono-, di- and triglycerides, and polyglycerol oleate or polyglycerol dioleate. For example, the di- and triglycerides have side chains with 16 to 18 carbons.

In particular examples, the large bioactive agent has a molecular weight of at least 200, and is sufficiently lipophilic that it has an octanol/water partitioning coefficient of at least 4. Particular examples of the bioactive agent are one or more of a steroid, a steroid antagonist, a non-steroidal anti-inflammatory agent, an anti-hypertensive agent, an antioxidant, an anti-seizure agent, an antibiotic (including anti-bacterial and anti-fungal agents), an antiviral agent, an anticancer agent, an anti-depressive agent, an enzyme, a coenzyme, a protein, a globulin, a vitamin, a retinoid, an immunological agent, a nucleotide, a lectin, or a growth factor.

In specifically disclosed examples, the polyphenol is one or more of:

a) resveratrol;

b) Polygonum cuspidatum extract;

c) green tea extract;

d) grape extract;

e) grape seed extract;

f) blackberry extract;

g) blueberry extract;

h) cranberry extract;

i) elderberry extract;

j) black current extract; or

k) oolong tea extract.

In other examples, the polyphenol or mixture of polyphenols includes one or more of a di- and/or trihydroxyphenyl compound chosen from:

a) catechins and substituted catechins;

b) flavanols;

c) flavondiols;

d) theaflavins;

e) thearubigens;

f) anthocyanidin and substituted anthocyanidins;

g) rutin and substituted rutin;

h) tannins; or

i) genistein and substituted genisten.

In particular examples, the polyphenol is a catechin and/or substituted catechin, such as one or more of epicatechin, epicatechin gall ate (ECG), epigallocatechin (EGC), epigallocatechin gallate (EGCG), or gallocatechin.

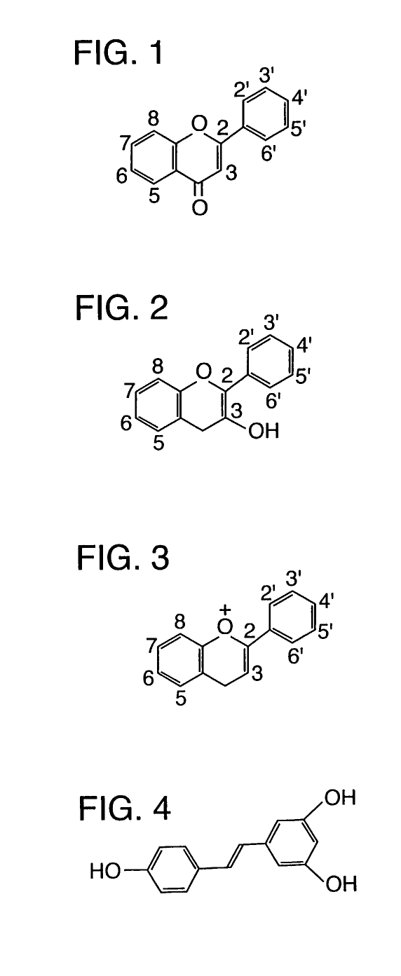

Among the polyphenols are the following classes, as noted in U.S. Pat. No. 6,099,854: flavonoids (a term often used to denote polyphenols in general, but more commonly in Europe to denote only the flavones), the flavanols, proanthocyanidins (also called procyanidols, procyanins, procyanidins and tannins) and anthocyanins. The flavones are compounds with a basic structure shown in FIG. 1 in which two benzene rings (A and B) are linked with a heterocyclic six member ring C containing a carbonyl group. Ring B can be joined in position 2 (as illustrated) to give a flavone or to position 3 to give an iso flavone. Hydroxylation can occur at positions 3, 5, 7 and 3′, 4′, 5′ to give compounds called flavonols. Typical examples of flavonols are: quercetin, (hydroxylated at positions 3, 5, 7, 3′, 4′), kaempferol (hydroxylated at positions 3, 5, 7, 4′), and myricetin (hydroxylated at positions 3, 5, 7, 3′, 4′, 5′). They can exist naturally as the aglycone or as O-glycosides (e.g. D-glucose, galactose, arabinose, rhamnose etc). Other forms of substitution such as methylation, sulphation and malonylation are also found.

The flavonols have a basic structure shown in FIG. 2. The two most common flavonols are catechin (hydroxyl groups positions 5, 7, 3′, 4′) and its stereo-isomer epi-catechin. The proanthocyanidins are polymers of catechin and/or epicatechin and can contain up to 8 units or more.

The anthocyanins are colored substances with a basic structure shown in FIG. 3. They are sometimes called anthocyanidins. Typical examples are: cyanidin (hydroxylated at positions 3, 5, 7, 3′, 4′), delphinidin (hydroxylated at positions 3, 5, 7, 3′, 4′, 5′) and pelargonidin (hydroxylated at positions 3, 5, 7, 3′). The hydroxyl groups are often glycosylated and/or methoxylated (e.g. malvidin at 3′, 5′).

Within the general term “polyphenols” are included the dihydroxy- or tri-hydroxy benzoic acids and the phytoalexins, a typical example of which is resveratrol (shown in FIG. 4).

In particular examples of the composition, the polyphenol comprises one or more flavonols, such as quercetin, kampferol, or myricetin. In these or other examples, the bioactive agent is Coenzyme Q10 in either its reduced form (ubiquinone) or oxidized form (ubiquinol), for example in its oxidized form. Alternatively, it can be present in both its reduced and oxidized forms. Particular examples of compositions include a polyphenol such as Polygonum cuspidatum extract and/or resveratrol.

Some examples of the composition may include an anti-oxidant other than the polyphenol or bioactive agent. Particular non-limiting examples of such an anti-oxidant include Vitamin A, Vitamin E (tocopherol), Vitamin K, Copper, Zinc, Iron, Selenium, beta-carotene, eriodictyol, carnosic acid, carnosol, rosmarrinic acid, caffeic acid, coumaric acid, cinnamic acid, Coenzyme Q, Probucol, astaxanthin, lycopene, alpha-lipoate, and urate.

In a particular disclosed example of the composition, the bioactive agent is Coenzyme Q (another term from ubiquinone), the polyphenol is Polygonum cuspidatum extract (a material rich in resvertrol), the lipid matrix comprises a mixture of refined soybean oil, with mono-, di- and triglycerides, and polyglycerol oleate and polyglycerol dioleate, and the composition comprises tocopherol as an anti-oxidant other than Coenzyme Q or the polyphenol. In other particular examples, the composition includes about 8-10% bioactive agent, less than about 1% polyphenol, and about 85-90% lipid matrix. In other embodiments, the composition further includes about 2-3% of an anti-oxidant other than the bioactive agent and polyphenol.

Also disclosed are methods of improving absorption of a high molecular weight, lipophilic, bioactive agent in the gastrointestinal tract, by orally administering to a subject the compositions described herein. In yet other embodiments, the method also includes a method of improving the stability of the composition prior to administration.

Examples of Formulations

The following formulations illustrate specific non-limiting examples of compositions with improved bioavailability and stability.

As illustrated in these non-limiting examples, particular embodiments of the compositions can have a ratio of gel oil to active ingredient of at least about 4:1, for example at least about 10:1 or at least about 30:1, and the polyphenol (or mixture of polyphenols) can be less than about 1% by weight of the composition. In particular examples, there is less than about 1 mg of the polyphenol component.

Bioavailability Test Methodology

In order to demonstrate the enhanced absorption of large, high molecular weight, lipophilic, bioactive compounds from a matrix containing a polyphenolic compound as described herein, a study was conducted on the absorption of CoQ10 dissolved in a rice bran oil matrix encapsulated in a soft gelatin capsule (Example 5, Table 6). This formula served as the standard since it is reported to exhibit the highest CoQ10 bioavailability of any product on the market today. That standard was compared to CoQ10 in a soybean oil matrix containing 640 micrograms of Polygonum cuspidatum Extract which contains resveratrol, a polyphenolic compound (Example 6, Table 6). A blood sample was taken from each test subject and analyzed for its CoQ10 level as well as low-density lipoprotein (LDL) level. Then the test subjects were randomly given 3 soft gel capsules (a total of 90 mg of CoQ10) of one of the two formulations (either the standard or the polyphenolic containing formulation) on the first day of the study. The amount of CoQ10 in the body was then measured regularly over the next thirty-six (36) hours by drawing blood samples and determining the amount of CoQ10 in those blood samples. The amount of CoQ10 present in the blood was then calculated as the peak plasma levels as well as the percentage of the baseline blood levels of CoQ10 present before the ingestion of this high molecular weight, lipophilic bioactive agent. Following a 10-day wash-out period, the test subjects were then given 3 soft gel capsules (90 mg) of the other formulation and the analysis of blood levels of CoQ10 was repeated.

Basal plasma levels of CoQ10 were determined on all volunteer subjects at 7:00 AM on days −20 and −10 after an 8-hour fast. The inclusion criteria were normal volunteer subjects 20 to 55 years of age with basal blood plasma CoQ10 levels between 0.70 and 0.85 μg/ml of blood plasma and blood plasma LDL levels below 130 mg/dl. The basal blood plasma levels of the test subjects are shown in Table 7 below. On the day of each study, the volunteers again reported to the testing laboratory at 7:00 AM in a fasting state. A catheter was placed in an appropriate vein of the forearm for the purpose of drawing blood samples. After an initial blood sample at 7:00 AM, 90 mg of the appropriate CoQ10 sample was ingested. Blood samples were then collected at two-hour intervals for 12 hours, at 24 hours (fasting) and at 36 hours. Between the 0- and 12-hour intervals, a low fat breakfast and lunch without CoQ10 was provided. All venous blood samples were cooled in ice, then centrifuged with the plasma separated, frozen at −50° C. The amount of CoQ10 in the various blood samples was then determined using HPLC according to the method of Morita and Folkers (Biochem. Biophy. Res.Comm. 191 (13): 950-954, 1993).

Results of the Bioavailability Study of Example Formulations 5 and 6

The results obtained by measuring the amount of CoQ10 in the blood serum of subjects administered the formulation without the polyphenolic (Example Formula 5) are shown in Table 8, including the mean values and the standard deviation at each assay point. The results obtained for the formulation with the polyphenolic (Example Formula 6) are similarly shown in Table 9. An examination of the data found in either of these tables reveals that the oral ingestion of each of these formulas results in an increase in the level of CoQ10 in the bloodstream. A statistical evaluation of the data found in Tables 8 and 9 shows that both formulations achieve statistically significant blood plasma levels within 4 hours following ingestion. As shown in Tables 10 and 11, these statistically significant levels are maintained throughout the 36-hour duration of this study for each of the formulations tested.

When the data for the two formulations are compared, the advantages of the polyphenolic compositions become clear. As shown in Table 12, Example Formula 6 (with polyphenols) not only yields higher blood plasma levels of CoQ10 within 4 hours following oral ingestion when compared to Example Formula 5 (without polyphenols), but those blood plasma levels are statistically significantly higher. Furthermore, those statistically higher blood plasma levels of CoQ10 are maintained throughout the 36-hour period following the administration of a single dose of this large, high molecular weight, lipophilic, bioactive agent. Additionally, as shown in Table 13, the administration of the formulation containing the polyphenolic (Example Formula 6) yields statistically significantly higher results in every parameter evaluated when compared to the formulation without the polyphenolic (Example Formula 5). In total, these results clearly demonstrate that this new technology delivers unique and unexpected bioavailability for large, high molecular weight, lipophilic, bioactive agents.

Table 13 also shows the peak blood plasma levels of CoQ10. In the presence of the polyphenolic, the peak plasma level found was 3.63 μg/ml of blood. This value is statistically higher than the peak plasma level found for the best commercial formulation available in the marketplace, namely the 2.72 μg/ml of blood value for Example Formula 5. Furthermore, this latter value is equivalent to the peak plasma value reported in the literature for the best formulation employing a soybean oil base as shown in Table 2, namely a peak plasma level of 2.84 μg/ml of blood. This comparison becomes important since the formulation containing the polyphenolic also employs a soybean oil base in which to dissolve the CoQ10. However, in order for the soybean oil based formulation reported in Table 2 to be judged as not statistically different from that of Example Formula 6, the standard deviation from the bioavailability test of the prior formula (as reported in Table 2) would have to be larger than 0.86. Given the fact that this test protocol yields standard deviations that are approximately one-sixth that large, it is believed that these 2 soybean oil formulations must be statistically different. Therefore, no direct comparison testing was deemed necessary.

Based upon this information, the presence of a polyphenolic in a oil based matrix is capable of enhancing the bioavailability of large, high molecular weight, lipophilic, bioactive agents from an orally ingestible, non-dry powder, dosage form. This phenomenon has not previously been reported.

Stability Test Methodology

Stability testing of formulations developed according to the technologies described herein was conducted on the soft gelatin capsules stored in high density polyethylene jars employing a polyethylene closure. No special precautions were employed to exclude atmospheric oxygen from the container surrounding the gelatin capsules.

Packages containing the soft gelatin capsules were stored under conditions recommended by the U.S. Food and Drug Administration guidelines for the determination of expiry dating of pharmaceutical preparations from accelerated stability. The formulations were stored for periods of up to 12 weeks under the following conditions:

1. 0° C.; ambient humidity

2. 25° C.; 60% relative humidity

3. 35° C.; 75% relative humidity

4. 40° C.; 75% relative humidity

Samples of the capsules were removed from the containers periodically throughout the storage period and evaluated for obvious change in coloration and for the visual examined. The capsules were then carefully open. The contents of the capsules were microscopically evaluated for the presence of crystals.

Results of the Bioavailability Study of Example Formulations 6 and 7

All capsules were found to be intact and virtually unchanged by visual examination at each evaluation point. The results of the microscopic examination at the conclusion of the accelerated 12-week storage period are shown in Table 15.

The accelerated storage results clearly indicate that although there are some small crystalline clumps of what appears to be the polyphenolic (Polygonum cuspidatum extract in this particular case) that is not solubilized by the formulation matrix, the number of crystals of CoQ10 present in Example Formulation 6 appear to be reduced and the crystals present are less highly aggregated when compared to the crystals of CoQ10 in Example Formulation 7. Therefore, not only does the presence of the polyphenolic reduce the number of crystals of the CoQ10 present in the oil matrix, but it also reduces the degree of aggregation of those crystals that are present. These results clearly demonstrate that the presence of the polyphenolic improves the stability of the formulation. Additionally, given the data presented previously regarding the bioavailability of powdered CoQ10, it is not surprising that the reduced number and size of the crystals of CoQ10 in the matrix of Example Formulation 6 clearly bears a relationship to the bioavailability of this formulation.

TABLE 1
Large, Lipophilic Bioactive Agents
(Listed by Class or Type of Bioactive Agent)

| Bioactive Agent Class | Ingredient | Molecular Weight |
|---|---|---|
| ACE Inhibitors | Candesartan cilexetril | 611 |
| | Gralapril Malate | 493 |
| Analgesics (including opiod | Fetanyl citrate | 529 |
| types) | Lidocaine | 243 |
| | Prilocaine | 220 |
| Antibiotic/Antifungal/Anti- | Clarithromycin | 748 |
| microbial Agents | Erythromycin | 862 |
| | (its salts and esters) | (as the ethyl |
| | | succinate) |
| | Clotrimazole | 345 |
| | Sparfloxacin | 392 |
| Anticancer Agent | Docetaxel | 862 |
| | Etoposide | 589 |
| | Lomustine | 234 |
| | Paclitaxel | 854 |
| | Teniposide | 657 |
| Anticonvulsants (also | Lorazepam | 321 |
| seditives) | Primidone | 218 |
| Antidiebetic Agent | Glimepilude | 491 |
| Antihypertensive Agents | Methylclothiazide | 347 |
| Antiinflammatory Agents | Budesonide | 431 |
| Calcium Channel Blockers | Felodipine | 384 |
| | Nisoldipine | 388 |
| | Nifedipine | 246 |
| | Mimodipine | 419 |
| Cardiac regulating agents | Digoxin | 781 |
| Digestive Aid Agents | Ursodiol | 393 |
| Enzyme Inhibitors | Zileuton | 237 |
| Hypnotic Agents | Estrzolam | 295 |
| Immunosupressive Agents | Cyclosporin | 1203 |
| | Tacrolimus | 822 |
| Lipid Inhibitors | Fenofibrute | 361 |
| Peptides | Cyclosporin | 1203 |
| Protease Inhibitors | Ritonavir | 721 |
| Steroid Antagonists | Bicalutamide | 430 |
| | Nilretamide | 317 |
| | Tamsulosin | 445 |
| | Testolactone | 300 |
| Steroids (including | Betamethasone | 477 |
| corticosteroids) | valerate | |
| | Flutacasone | 501 |
| | proprionate | |
| Vitamin | Vitamin D3 | 416 |
| | (cholecalciferol) | |

TABLE 2
Peak Plasma Levels of CoQ10 from Administration
of 100 mg of a Dry Powder Formulation

| | Plasma CoQ10 Levels (μg/ml) | |
|---|---|---|
| Author/Country (Year of Publication) | Baseline¹ | Peak |
| Judy/USA (1984) | 0.66 | 1.68 |
| Vaufraechen/USA (1984) | 1.10 | 1.72 |
| Langsjeon/USA (1984) | 0.97 | 2.44 |
| Judy/USA (1984) | 0.56 | 1.76 |
| Mortensen/USA (1984) | 0.66 | 1.51 |
| Wilson/USA (1984) | 0.79 | 1.87 |
| Yamaguchi/Japan (1985) | 0.74 | 1.60 |
| Takahashi/Japan (1985) | 0.41 | 1.86 |
| Folkers/USA (1985) | 0.67 | 2.00 |
| Judy/USA (1990) | 0.67 | 1.77 |
| Littarru/Italy (1990) | 0.85 | 1.63 |
| Judy/USA (1991) | 0.60 | 2.17 |

¹Plasma levels of CoQ10 prior to administration of the formulation.

TABLE 3
Peak Plasma Levels of CoQ10 from Administration of 100 mg
of a Soybean Oil Gelatin Capsule Formulation

| | |
|---|---|
| Author/Country | Plasma CoQ10 Levels (μg/ml) |

| (Year of Publication) | Baseline¹ | Peak |
|---|---|---|
| Judy/USA (1984) | 0.61 | 2.65 |
| Langsjeon/USA (1984) | 0.71 | 2.26 |
| Frustace/Italy (1985) | 0.86 | 2.84 |
| Ota/Japan (1985) | 0.56 | 2.60 |
| Schneebege/Germany (1985) | 0.92 | 2.64 |
| Folkers/USA (1985) | 0.67 | 2.48 |
| Judy/USA (1986) | 0.51 | 2.70 |
| Langsjeon/USA (1990) | 0.83 | 2.54 |
| Judy/USA (1990) | 0.62 | 2.66 |
| Judy/USA (1993) | 0.65 | 2.48 |
| Weiss/Denmark (1993) | 0.81 | 2.46 |
| Folkers/USA (1994) | 0.98 | 2.58 |

¹Plasma levels of CoQ10 prior to administration of the formulation.

TABLE 4
Formulation of Example 1

| Ingredients | Example 1 |
|---|---|
| GelOil SC [composed of refined soybean oil; | 2 to 5000 mg |
| mono-, di- and/or triglycerides (of 16 to 18 | |
| carbons), polyglycerol oleate and/or dioleate]¹ | |
| Lipophilic Bioactive Agent(s) | 1 ng to 1000 mg |
| Polyphenolic Compound(s) | 1 μg to 500 mg |
| Tocopherol or Mixed Tocopherols | 1 μg to 500 mg |
| ¹GelOil SC is a proprietary blend of Soft Gel Technologies, Los Angeles, CA | |
| Procedure: Heat the GelOil SC to 25° to 35° C. in a vessel under vacuum. Remove the vacuum, quickly add the lipophilic bioactive agent(s), the polyphenolic compound(s), and the tocopherol to the heated GelOil SC. Reinstate the vacuum to prevent oxidation. Blend and continuously stir mixture until all ingredients are dissolved in the GelOil SC. Cool the mixture to 25° to 30° C. | |
| # Remove vacuum and blanket mixture with nitrogen. Encapsulate the mixture into soft gelatin capsules. | |

TABLE 5
Formulations for Specific Large, High Molecular Weight, Lipophilic,
Bioactive Agents

| | Examples | | | | | | | |
|---|---|---|---|---|---|---|---|---|
| Ingredients | 1 | | 2 | | 3 | | 4 | |
| GelOil SC¹ | 337 | mg | 337 | mg | 287 | mg | 317 | mg |
| Amphotericin B | 10 | mg | — | | — | | — | |
| (antibiotic) | | | | | | | | |
| Nystatin (antifungal) | — | | 10 | mg | — | | — | |
| Dexanabinol (neuropro- | — | | — | | 60 | mg | — | |
| tective drug) | | | | | | | | |
| Coenzyme Q10 | — | | — | | — | | 30 | mg |
| Polyphenolic | 1000 | μg | 1000 | μg | 1000 | μg | 1000 | μg |
| Compound(s) | | | | | | | | |
| Tocopherol or Mixed | 10 | mg | 10 | mg | 10 | mg | 10 | mg |
| Tocopherols | | | | | | | | |
| ¹GelOil SC is a proprietary blend of Soft Gel Technologies, Los Angeles, CA | | | | | | | | |
| Procedure: Heat the GelOil SC to 25° to 35° C. in a vessel under vacuum. Remove the vacuum, quickly add the lipophilic bioactive agent (Amphotericin B, Nystatin, Dexanabinol, or Coenzyme Q10), the polyphenolic compound(s), and the tocopherol to the heated GelOil SC. Reinstate the vacuum to avoid oxidation. Blend and continuously stir mixture until all ingredients are dissolved in the GelOil SC. | | | | | | | | |
| # Cool the mixture to 25° to 30° C. Remove vacuum and blanket mixture with nitrogen. Encapsulate the mixture into soft gelatin capsules. | | | | | | | | |

TABLE 6
Formulations for Bioavailability Testing (Examples 5 and 6)

Examples

| Ingredients | 5 | 6 |
|---|---|---|
| GelOil¹ | 318 mg | — |
| GelOil SC² | — | 317 mg |
| Coenzyme Q10 | 30 mg | 30 mg |
| Polygonum cuspidatum Extract | — | 640 μg |
| Mixed Tocopherol | 10 mg | 10 mg |
| ¹GelOil is a proprietary blend of Soft Gel Technologies, Los Angeles, CA [GelOil is a mixture of Rice Bran Oil, Yellow Beeswax, and Beta Carotene] | | |
| ²GelOil SC is a proprietary blend of Soft Gel Technologies, Los Angeles, CA | | |
| Procedure: Heat the GelOil SC or GelOil to 25° to 35° C. in a vessel under vacuum. Remove the vacuum, quickly add the Coenzyme Q10, the Polygonum cuspidatum Extract, and the tocopherol to the heated GelOil SC or GelOil. Reinstate the vacuum to prevent oxidation. Blend and continuously stir mixture until all ingredients are dissolved in the GelOil SC or GelOil. Cool the mixture to 25° to 30° C. | | |
| # Remove vacuum and blanket mixture with nitrogen. Encapsulate the mixture into soft gelatin capsules. | | |

TABLE 7
Basal CoQ10 and LDL Levels for Test Subjects

| | |
|---|---|
| CoQ10 Levels¹ | |
| In Test Number | Serum LDL Levels |

| Test Subject No. | Age | 1 | 2 | Start of Test² |
|---|---|---|---|---|
| 1 | 26 | 0.76 | 0.78 | 102 |
| 2 | 32 | 0.74 | 0.80 | 122 |
| 3 | 20 | 0.80 | 0.83 | 110 |
| 4 | 51 | 0.77 | 0.81 | 94 |
| 5 | 62 | 0.78 | 0.76 | 119 |

¹Basal CoQ10 levels in the blood prior to initiation of the test (in μg/ml blood)
²Basal LDL (low density lipoprotein) levels in the blood prior to the initiation of the test (in μg/ml blood)

TABLE 8
Plasma Blood Levels of CoQ10 from Formulations without Polyphenols
(Example Formula 5)
CoQ10 Levels¹ Measured × Hours After Dosing

| Subject | | | | | | | | | | |
|---|---|---|---|---|---|---|---|---|---|---|
| No. | −1 | 0 | 2 | 4 | 6 | 8 | 10 | 12 | 24 | 36 |
| 1 | 0.76 | 0.75 | 0.78 | 1.19 | 2.66 | 2.20 | 1.73 | 1.14 | 1.61 | 1.35 |
| 2 | 0.75 | 0.74 | 0.76 | 1.22 | 2.65 | 2.14 | 1.97 | 1.45 | 1.56 | 1.21 |
| 3 | 0.78 | 0.78 | 0.76 | 1.43 | 2.89 | 2.31 | 1.69 | 1.34 | 1.41 | 1.23 |
| 4 | 0.79 | 0.80 | 0.78 | 1.34 | 2.84 | 2.15 | 1.78 | 1.67 | 1.58 | 1.34 |
| 5 | 0.74 | 0.73 | 0.76 | 1.23 | 2.57 | 2.00 | 1.66 | 1.50 | 1.63 | 1.39 |
| Average | 0.76 | 0.76 | 0.77 | 1.28 | 2.72 | 2.16 | 1.77 | 1.42 | 1.56 | 1.30 |
| S.D.² | 0.02 | 0.03 | 0.01 | 0.10 | 0.14 | 0.11 | 0.12 | 0.20 | 0.09 | 0.08 |

¹CoQ10 levels in the blood (in μg/ml blood)
²S.D. is the standard deviation.

TABLE 9
Plasma Blood Levels of CoQ10 from Formulation with Polyphenols
(Example Formula 6)
CoQ10 Levels¹ Measured × Hours After Dosing

| Subject | | | | | | | | | | |
|---|---|---|---|---|---|---|---|---|---|---|
| No. | −1 | 0 | 2 | 4 | 6 | 8 | 10 | 12 | 24 | 36 |
| 1 | 0.75 | 0.76 | 0.78 | 1.56 | 3.51 | 2.40 | 2.31 | 1.66 | 1.78 | 1.44 |
| 2 | 0.74 | 0.73 | 0.74 | 1.69 | 3.76 | 2.86 | 2.30 | 1.87 | 2.00 | 1.65 |
| 3 | 0.78 | 0.81 | 0.81 | 1.58 | 3.69 | 2.34 | 2.40 | 1.56 | 1.72 | 1.44 |
| 4 | 0.80 | 0.78 | 0.78 | 1.76 | 3.76 | 2.87 | 1.31 | 1.66 | 1.85 | 1.45 |
| 5 | 0.76 | 0.76 | 0.76 | 1.67 | 3.45 | 2.66 | 2.45 | 1.46 | 1.73 | 1.34 |
| Average | 0.77 | 0.77 | 0.77 | 1.65 | 3.63 | 2.63 | 2.15 | 1.64 | 1.82 | 1.46 |
| S.D.² | 0.02 | 0.03 | 0.03 | 0.08 | 0.15 | 0.25 | 0.48 | 0.15 | 0.11 | 0.11 |

¹CoQ10 levels in the blood (in μg/ml blood)
²S.D. is the standard deviation.

TABLE 10
Statistical Comparison of Blood Plasma Levels of CoQ10 (μg/ml of
Blood) of Pre-treatment to Post-treatment in the Absence of the
Polyphenolic

| | Post-treatment | | Pre-treatment | | |
|---|---|---|---|---|---|
| | Blood Levels | | Blood Levels¹ | | Statistical |
| Eval. Time | Average | Std. Dev.² | Average | Std. Dev.² | Significance³ |
| 2 hrs. | 0.77 | 0.03 | 0.77 | 0.03 | No |
| 4 hrs. | 1.28 | 0.10 | 0.77 | 0.03 | Yes |
| 6 hrs. | 2.72 | 0.14 | 0.77 | 0.03 | Yes |
| 8 hrs. | 2.16 | 0.11 | 0.77 | 0.03 | Yes |
| 10 hrs. | 1.77 | 0.12 | 0.77 | 0.03 | Yes |
| 12 hrs. | 1.42 | 0.20 | 0.77 | 0.03 | Yes |
| 24 hrs. | 1.56 | 0.09 | 0.77 | 0.03 | Yes |
| 36 hrs. | 1.30 | 0.08 | 0.77 | 0.03 | Yes |

¹Blood levels of CoQ10 prior to dosing
²Std. Dev. stands for standard deviation.
³Statistically significant at the 95% confidence level using a student t-test.

TABLE 11
Statistical Comparison of Blood Plasma Levels of CoQ10 (μg/ml
of Blood) of Pre-treatment to Post-treatment in the Presence of the
Polyphenolic

| | Post-treatment | | Pre-treatment | | |
|---|---|---|---|---|---|
| | Blood Levels | | Blood Levels¹ | | Statistical |
| Eval. Time | Average | Std. Dev.² | Average | Std. Dev.² | Significance³ |
| 2 hrs. | 0.77 | 0.03 | 0.77 | 0.03 | No |
| 4 hrs. | 1.65 | 0.08 | 0.77 | 0.03 | Yes |
| 6 hrs. | 3.63 | 0.15 | 0.77 | 0.03 | Yes |
| 8 hrs. | 2.63 | 0.25 | 0.77 | 0.03 | Yes |
| 10 hrs. | 2.15 | 0.48 | 0.77 | 0.03 | Yes |
| 12 hrs. | 1.64 | 0.15 | 0.77 | 0.03 | Yes |
| 24 hrs. | 1.82 | 0.12 | 0.77 | 0.03 | Yes |
| 36 hrs. | 1.46 | 0.11 | 0.77 | 0.03 | Yes |

¹Blood levels of CoQ10 prior to dosing
²Std. Dev. stands for standard deviation.
³Statistically significant at the 95% confidence level using a student t-test.

TABLE 12
Statistical Comparison of Blood Plasma Levels of CoQ10 (μg/ml of
Blood) in the Presence and Absence of the Polyphenolic

| | Without | | | | |
|---|---|---|---|---|---|
| | Polyphenolic¹ | | With Polyphenolic² | | Statistically |
| Eval. Time | Average | Std. Dev.³ | Average | Std. Dev.³ | Significant⁴ |
| −1 hrs. | 0.76 | 0.02 | 0.77 | 0.02 | No |
| 0 hrs. | 0.76 | 0.03 | 0.77 | 0.03 | No |
| 2 hrs. | 0.77 | 0.01 | 0.77 | 0.03 | No |
| 4 hrs. | 1.28 | 0.10 | 1.65 | 0.08 | Yes |
| 6 hrs. | 2.72 | 0.14 | 3.63 | 0.15 | Yes |
| 8 hrs. | 2.16 | 0.11 | 2.63 | 0.25 | Yes |
| 10 hrs. | 1.77 | 0.12 | 2.15 | 0.48 | Yes |
| 12 hrs. | 1.42 | 0.20 | 1.64 | 0.15 | Yes |
| 24 hrs. | 1.56 | 0.09 | 1.82 | 0.12 | Yes |
| 36 hrs. | 1.30 | 0.08 | 1.46 | 0.11 | Yes |

¹Formulation without Polyphenolic - Example Formula 5
²Formulation with Polyphenolic - Example Formula 6
³Std. Dev. stands for standard deviation.
⁴Statistically significant at greater than the 90% confidence level using a student t-test.

TABLE 13
Statistical Comparison of Other Parameters from Blood Plasma
Levels Of CoQ10 in the Presence and Absence of a Polyphenolic
Compound

| | Without | | With | | |
|---|---|---|---|---|---|
| | Polyphenolic¹ | | Polyphenolic² | | |
| | | Std. | | Std. | Statistically |
| Parameter | Average | Dev.³ | Average | Dev.³ | Significant⁴ |
| Peak Plasma Level | 2.72 | 0.14 | 3.63 | 0.15 | Yes |
| (μg/ml) | | | | | |
| Percentage Increase in | 258 | 7.46 | 374 | 25.6 | Yes |
| Peak Plasma Levels | | | | | |
| Peak Change (μg/ml) | 1.97 | 0.11 | 2.66 | 0.30 | Yes |
| Peak Absorption Rate | 18.2 | 3.78 | 27.0 | 9.20 | Yes |
| (μg/min) | | | | | |
| Peak Amount Absorbed | 6.56 | 1.36 | 8.98 | 2.30 | Yes |
| (mg) | | | | | |
| % of Dose Absorbed | 7.30 | 1.48 | 9.98 | 2.60 | Yes |
| Distribution Rate | 5.05 | 2.10 | 19.01 | 4.08 | Yes |
| (μg/min) | | | | | |
| AUC for 0-36 hr | 31.8 | 2.20 | 43.5 | 6.20 | Yes |
| (μg/ml · hr)⁵ | | | | | |

¹Formulation without Polyphenolic - Example Formula 5
²Formulation with Polyphenolic - Example Formula 6
³Std. Dev. stands for standard deviation.
⁴Statistically significant at the 95% confidence level using a student t-test.
⁵Area under the curve for absorption of CoQ10 between 0 and 36 hours in μg/ml · hr.

TABLE 14
Formulations for Stability Testing (Examples 6 and 7)

Examples

| Ingredients | 6 | 7 |
|---|---|---|
| GelOil SC¹ | 317 mg | 318 mg |
| Coenzyme Q10 | 30 mg | 30 mg |
| Polygonum cuspidatum Extract | 640 μg | — |
| Mixed Tocopherol | 10 mg | 10 mg |

¹GelOil SC is a proprietary blend of Soft Gel Technologies, Los Angeles, CA
Procedure: Heat the GelOil SC to 25° to 35° C. in a vessel under vacuum. Remove the vacuum, quickly add the Coenzyme Q10, the Polygonum cuspidatum Extract, and the tocopherol to the heated GelOil SC. Reinstate the vacuum to prevent oxidation. Blend and continuously stir mixture until all ingredients are dissolved in the GelOil SC. Cool the mixture to 25° to 30° C. Remove vacuum and blanket mixture with nitrogen. Encapsulate the mixture into soft gelatin capsules.

TABLE 15
Results of Microscopic Evaluation of the Matrix Contents of the
Soft Gel Capsules of Example Formulations 6 and 7 After 12 Weeks
of Storage

| | Microscopic Evaluation Results | |
|---|---|---|
| Storage Condition | Formula 6 | Formula 7 |
| 0° C.; | A homogenous mixture of | A homogenous mix- |
| ambient RH¹ | small brown to purple | ture of fairly large |
| | crystal (Polygonum cupidatum | yellow CoQ10 crystals |
| | extract) and small yellow | without clumping in an |
| | CoQ10 crystals without | oily matrix. |
| | clumping in an oily matrix. | |
| | These CoQ10 crystals are | |
| | less than one-half the size | |
| | found in Formula 7 stored | |
| | at this condition. | |
| 25° C.; 60% RH | A homogenous mixture of | A homogenous mix- |
| | small brown to purple | ture of fairly large |
| | crystals (Polygonum | yellow CoQ10 crystals |
| | cupidatum extract) | without clumping in an |
| | and small yellow CoQ10 | oily matrix. |
| | crystals without clumping | |
| | in an oily matrix. These | |
| | CoQ10 crystals are less | |
| | than one-half the size | |
| | found in Formula 7 stored | |
| | at this condition. | |
| 35° C.; 60% RH | A homogeneous mixture of | A homogeneous mix- |
| | small yellow CoQ10 with | ture of yellow CoQ10 |
| | larger cloud-like clumps | crystals. These crystals |
| | of brown to purple crystals | are not as dense or as |
| | (Polygonum cupidatum | large as those found |
| | extract). The CoQ10 crystals | at 0° or 25° C. |
| | are smaller than those found | for this formula. |
| | in Formula 7 at this | |
| | condition. | |
| 40° C.; 75% RH | A homogeneous mixture of | A homogeneous mix- |
| | small yellow CoQ10 with | ture of yellow CoQ10 |
| | larger clumps of brown to | crystals. These crystals |
| | purple crystals | are not as dense or as |
| | (Polygonum cupidatum | large as those found |
| | extract). | at 0° or 25° C. |
| | | for this formula. |

¹RH - relative humidity

This specification has provided several detailed examples of the invention, which are not intended to be limiting. Rather, these examples are provided to illustrate some of the embodiments which come within the scope of the following claims.

What is claimed is:

1. An oral dosage composition of a high molecular weight, lipophilic, bioactive agent, comprising:
a biologically effective amount of the bioactive agent;
a lipid matrix in which the bioactive agent is suspended; and
a sufficient amount of a polyphenol to improve gastrointestinal absorption of the bioactive agent when the dosage form is orally administered.

2. The composition of claim 1, wherein the lipid matrix comprises a triglyceride matrix that is a liquid at body temperature.

3. The composition of claim 2, wherein the triglyceride matrix comprises a soybean lipid matrix.

4. The composition of claim 1, wherein the bioactive agent has a molecular weight of at least 200.

5. The composition of claim 4, wherein the bioactive agent comprises one or more of a steroid; a steroid antagonist; a non-steroidal anti-inflammatory agent; an anti-hypertensive agent; an antioxidant; an anti-epileptic agent; an antibiotic; an antiviral agent; an anticancer agent; a neuroprotective agent; an anti-depressive agent; an enzyme; a coenzyme; a protein; a globulin; a vitamin; a retinoid; an immunological agent; a nucleotide; a lectin; or a growth factor.

6. The composition of claim 3, wherein the lipid matrix comprises a mixture of refined soybean oil; mono-, di- and triglycerides, and polyglycerol oleate and/or polyglycerol dioleate.

7. The composition of claim 6, wherein the di- and triglycerides are glycerides having side chains with 16 to 18 carbons.

8. A method of claim 1 wherein the polyphenol comprises one or more of:
l) resveratrol;
m) Polygonum cuspidatum extract;
n) green tea extract;
o) grape extract;
p) grape seed extract;
q) blackberry extract;
r) blueberry extract;
s) cranberry extract;
t) elderberry extract;
u) black current extract; or
v) oolong tea extract.

9. The method of claim 1 wherein the polyphenol or mixture of polyphenols comprises one ore more of a di- and/or trihydroxyphenyl compound chosen from:
a) catechins and substituted catechins;
b) flavanols;
c) flavondiols;
d) theaflavins;
e) thearubigens;
f) anthocyanidin and substituted anthocyanidins;
g) rutin and substituted rutin;
h) tannins; or
i) genistein and substituted genisten.

10. The composition of claim 9, wherein the polyphenol comprises a catechin and/or substituted catechin.

11. The composition of claim 10, wherein the catechin and/or substituted catechin comprises one or more of epicatechin, epicatechin gallate (ECG), epigallocatechin (EGC), epigallocatechin gallate (EGCG), or gallocatechin.

12. The composition of claim 9, wherein the polyphenol comprises one or more flavonols.

13. The compsition of claim 12, wherein the flavonol comprises one or more of quercetin, kampferol, or myricetin.

14. The composition of claim 1, wherein the bioactive agent comprises a Coenzyme Q in either its reduced form (ubiquinone) or oxidized form (ubiquinol).

15. The composition of claim 8, wherein the polyphenol comprises Polygonum cuspidatum extract.

16. The composition of claim 8, wherein the polyphenol comprises resveratrol.

17. The composition of claim 1, further comprising an anti-oxidant other than the polyphenol or bioactive agent.

18. The composition of claim 17, wherein the anti-oxidant comprises one or more of Vitamin A, Vitamin E (tocopherol), Vitamin K, Copper, Zinc, Iron, Selenium, beta-carotene, eriodictyol, carnosic acid, carnosol, rosmarrinic acid, caffeic acid, coumaric acid, cinnamic acid, Coenzyme Q, Probucol, astaxanthin, lycopene, alpha-lipoate, and urate.

19. The composition of claim 1, wherein the bioactive agent comprises Coenzyme Q10, the polyphenol comprises Polygonum cuspidatum extact, and the lipid matrix comprises a mixture of refined soybean oil; mono-, di- and triglycerides, and polyglycerol oleate and/or polyglycerol dioleate.

20. The composition of claim 19, further comprising an anti-oxidant other than the Coenzyme Q10 or the polyphenol.

21. The composition of claim 20, wherein the anti-oxidant comprises tocopherol.

22. The composition of claim 1, comprising about 8-10% bioactive agent, less than about 1% polyphenol, and about 85-90% lipid matrix.

23. The composition of claim 1, further comprising 2-3% of an anti-oxidant other than the bioactive agent and polyphenol.

24. A method of improving absorption of a high molecular weight, lipophilic, bioactive agent in the gastrointestinal tract, the method comprising orally administering to a subject the composition of claim 1.

25. The method of claim 24, comprising administering the composition of claim 2.

26. The method of claim 25, comprising administering the composition of claim 3.

27. The method of claim 26, comprising administering the composition of claim 4.

28. The method of claim 27, comprising administering the composition of claim 5.

29. The method of claim 28, comprising administering the composition of claim 6.

30. The method of claim 29, comprising administering the composition of claim 17.

31. The method of claim 24, comprising administering the composition of claim 19.

32. The composition of claim 18, wherein the anti-oxidant comprises a pharmaceutically acceptable salt or ester of the anti-oxidant.

33. The composition of claim 21, wherein the tocopherol comprises a pharmaceutically acceptable ester of tocopherol.
